(19) United States
(12) Patent Application Publication
(10) Pub. No.: US 2015/0313566 A1
(43) Pub. Date: Nov. 5, 2015

(54) Determining the Spatial Position and Orientation of the Vertebrae in the Spinal Column

(71) Applicant: DIERS ENGINEERING GMBH, Wiesbaden (DE)

(72) Inventors: Helmut DIERS, Schlangenbad (DE); Christian DIERS, Wiesbaden (DE); Carsten DIERS, Wiesbaden (DE)

(21) Appl. No.: 14/646,485

(22) PCT Filed: Nov. 18, 2013
(86) PCT No.: PCT/EP2013/074089

§ 371 Date: May 21, 2015

(30) Foreign Application Priority Data
Nov. 23, 2012 (DE) 10 2012 111 385.8

(51) Int. Cl. A61B 6/00; G06T 7/00

(52) U.S. Cl. CPC A61B 6/5247; A61B 6/505; G06T 7/003; G06T 7/0044; G06T 2207/10116; G06T 2207/10132; G06T 2207/30012; G06T 2207/30204

(57) ABSTRACT

The invention relates to a method for determining the spatial position and orientation of the vertebrae hi a spinal column, comprising the following steps: taking at least one X-ray image of at least part of the spinal column; simultaneous recording of surface data (30) of at least one part of the back by means of an optical method; determining the position of elements in the bone structure by means of the X-ray image; determining the position of distinct elements (40) in the surface data; determining anatomical fixed points; superimposing the at least one X-ray taken and the surface data recorded by means of the anatomical fixed points; calculating a three-dimensional model (50) from elements of the bone structure from the surface data and the at least one X-ray image, wherein the 50 model contains the position and orientation of the vertebrae, the progression (55) of the spinal column and the spinal processes, as well as the shift (60) of the spinal process progression and the spinal column progression. The present, adapted model enables additional X-ray images during check-ups to be avoided, even in patients with severe deformation of the spinal column (e. g. scoliosis).

### SCOPE OF THE INVENTION

The invention relates to a method and a device for determining the spatial position and orientation of the pelvis and/or the bone structures of the shoulder-arm region and/or the vertebrae of a spinal column of a vertebrate. Such methods and devices are used primarily for imaging the internal and external structure of the human or animal body for diagnostic purposes.

### STATE OF THE ART

X-ray imaging systems are used primarily for representing the bone structures and the skeletal system of the human body. An important application in this case is also the representation of the spinal column at various recording layers. One problem with X-ray technology is X-ray absorption by the human body during radiography, thereby increasing the risk of cancer. Scoliosis patients are particularly affected by this because, during the growth phase, the condition generally requires a very high number of X-rays in clinical checkups. Studies by Doody et al. [1] in the United States show that the subsequent cancer rate is many times higher among scoliosis patients compared to the normal population. Although newer techniques do indeed allow a reduction of the X-ray dose, ultimately, however, the increased cancer risk associated with radiography remains. In addition, when using the X-ray technique, the rotation (rotation about the vertical axis) of each vertebra of the spinal column cannot be determined, or only inadequately, because the image only exists in the form of a two-dimensional projection.

Optical 3D surface measurement systems are radiation-free (in the medical sense), i.e. they do not require ionising radiation, and are used in particular for the measurement of human posture. The University of Munster has developed both the technique of video rasterstereography as well as a method based on this [2] to allow model-like reconstruction of the spinal column. This system was made available to many scoliosis patients for radiation-free checkups. The patent EP 1 718 206 B1, which is incorporated into this description by reference, describes newer developments that also allow a functional model-like representation of the spinal column. This also makes it possible to use the radiation-free method extensively in diagnosis and checkups in other applications. The use of X-rays may be reduced through optical surface measurement, and the applied X-ray dosage, as well as the possible risk of cancer, may be reduced.

In addition to its use with scoliosis patients, the desire for radiation-free methods is also increasing for checkups, for example, before and after surgery, in rehabilitation, physiotherapy, etc.

However, optical surface measurement methods suffer from limitations in the model-like reconstruction of the spinal column in the presence of severe deformation of the back and the spinal column [3], whereby the accuracy, and thus unambiguous determination of the shape and position of the spinal column in patients, decreases [4].

In the case of patients with moderate deformation of the spinal column, the use of X-rays and checkups through optical measurement techniques, including reconstruction of the spinal column, have become standard practice. Furthermore, there are procedures that make possible the scanning of X-ray images for inclusion in the measurement result of an optical surface measurement. It is hardly possible to carry out mutually independent execution of X-ray imaging and optical surface measurements for reproducible positioning and posture of the patient during the recording. In the case of recording while standing, the body exhibits a natural fluctuation that occurs on average within a cycle time of about 5 seconds, and whose amplitude can be up to 30 mm. As the average X-ray recording time for an area recording takes up to 1 second (depending on the volume of the patient), the possible fluctuation amplitude is up to 6 mm. Furthermore, in the case of X-ray recording, patient positioning is not uniformly regulated or standardised. X-ray recording is also characterised by variable magnification factors in the detected image due to the existing beam geometry. A combination or mapping of measurement results of the two measurement methods is, therefore, generally subject to error.

Checkups in the case of severe scoliosis can, therefore, only be carried out including errors when using the optical surface measurement method. Scoliosis, here, refers to a lateral curvature of the spinal column with simultaneous rotation of the vertebrae, which cannot be corrected by the use of the muscles. Thus, the X-ray remains the preferred method for this group of patients, and there is thus no reduction of the X-ray dose.

### OBJECT

The object of the invention is to provide a method and a device in which the disadvantages of the prior art are minimised.

### SOLUTION

This object is solved by the inventions having the features of the independent claims. Advantageous developments of the inventions are characterised in the dependent claims. The wording of all the claims is hereby incorporated by reference in the content of this description. The inventions also include all sensible and, in particular, all mentioned combinations of independent and/or dependent claims.

Individual method steps are described below in detail. The steps need not necessarily be performed in the order presented, while the method to be outlined may also have further unspecified steps.

To solve the object, a method for determining the spatial position and orientation of the pelvis and/or the bone structures of the shoulder-arm region and/or the vertebrae of a spinal column of a vertebrate, is proposed and comprises the following steps:

a) recording of at least one X-ray image of at least part of the spinal column and/or the pelvis and/or the bone structures of the shoulder-arm region, for example a top view of the spine from the front or back, or a side view;

b) recording of surface data of at least part of the back of the vertebrate by means of an optical (i.e. with visible or infrared light, i.e. with wavelengths between 380 and 780 or 780 and 1000 nm) or ultrasound method (i.e. time of flight measurement);

c) wherein steps a) and b) are performed simultaneously with a maximum time interval of one second, corresponding to the maximum X-ray recording time, preferably 0.5 seconds, more preferably 0.3 seconds, even more preferably 0.1 seconds, or most preferably 0.05 seconds; the time interval is referenced to the beginning of each recording;

d) determining the position of elements of the bone structure, such as bones or certain parts or areas or edges of the bones, or joints, by means of the X-ray distinct image;

e) determining the position of distinct elements of the surface structure in the surface data, for example, elevations caused by the ends of the spinous processes of the vertebrae;

f) wherein the position of elements of the bone structure is deduced from the position of the distinct elements of the surface structure;

g1) determining matching elements of the bone structure from the at least one X-ray image and from the surface data as anatomical fixed points; or

g2) determining anatomical fixed points by means of markers on the back of the vertebrate, wherein the markers are selected such that they are visible both in the surface data as well as on the X-ray image;

h) superimposing the at least one recorded X-ray image and the surface data by means of the anatomical fixed points;

i) calculating a three-dimensional model of elements of the bone structure from the surface data and the at least one X-ray image, wherein the model includes:

i1) the position of the vertebrae and/or of the pelvis, i.e. three spatial coordinates, and/or

i2) the curvature of the spinal column and/or of the spinous processes and/or

i3) the orientation of the individual vertebrae and/or the pelvis, i.e. three angles of orientation (sagittal, lateral as well as rotational), and/or

i4) the displacement of the spinous process progression and the spinal column progression and/or

i5) the location and orientation of the shoulder blades and/or the bone structures of the shoulder-arm region.

The method is usually provided for use with human beings, but can be generally applied to other organisms with a spinal column, as long as the corresponding region of the back is accessible for optical or ultrasound measurement.

The at least one X-ray image is recorded with X-rays. This involves electromagnetic waves with photon energies between 50 and 150 keV, which correspond to wavelengths between 2.5 and 0.8*10⁻¹¹ m (8 to 25 pm).

The anatomical fixed points, which, for example, may be selected elements of the bone structure, serve to align the X-ray recording and the surface data. The intersection of the at least one X-ray image and the elements of the bone structure obtained from the surface data may be used, for example, as a selection criterion.

Three orientation angles are determined for each vertebra based on the position of the vertebrae and the position of the spinous processes. This enables accurate modelling of the entire spinal column, whereby the actual properties of the individual vertebrae are taken into account.

The integration of both measurement methods in a recording system and the simultaneous implementation of both measurements solves the existing problem. Differences in posture occurring between the two methods may be excluded by the simultaneous recording procedure, whereby the re-calculation of the magnification-reduction factors from the X-ray image is possible with knowledge of the surface parameters. Radiation-free optical or ultrasound surface measurement may be performed in checkups, even in the case of severe deformities, by knowing the starting position, i.e. the exact position and shape of the spinal column or the bone structures. In this case, the sequence shots with the radiation-free surface measurement are each respectively related to the output images or the output data and the respective deviations.

Until now in the case of dynamic and/or functional measurement methods, such as in video gait analysis, individual marker points have only been manually applied to the skin surface and analysed during movement. By using the dynamic optical surface measurement (EP 1 718 206 B1), it is possible to document and analyse entire area-wide distortions in gait and functional analyses. There is also a wish to be as realistic as possible in integrating the bone structure in the movement patterns and thus to determine them. In order to obtain accurate knowledge of the original shape and position of the osseous system, it is desirable to determine this with reference to an X-ray recording and to align this with an optical surface measurement method. The simultaneous performance of X-ray recording and surface measurement offers a means to this end.

Elements of the bone structure are advantageously determined as: a) the spinous processes and the pedicles of the vertebrae of the spinal column and/or b) from the pelvic region, the sacrum, the upper edge of the ilium and the anterior and/or posterior superior iliac spine and/or c) the clavicle and the acromioclavicular joint and/or d), the shoulder blades and/or e) the shoulder blade edges.

It is also advantageous when the distinct elements in the surface data are determined through analysis of surface properties, wherein

a) curvatures and/or symmetries in the surface data are calculated; and

b) the calculation of the curvatures and/or symmetries includes the fulfilment of certain predetermined conditions, which at least

b1) either describe the curvature or the symmetry of the surface, and

b2) either describe the relative position, bending, twisting or equidistance of the vertebrae of the spinal column.

It is advantageous if the X-ray image is scaled in the method, in order to generate a uniform true-to-scale representation of the surface data and X-ray data. This allows a common analysis of the data with minimum deviations.

In an advantageous development of the method, at least further optical or ultrasound recordings are performed (so-called checkups) at a later point in time, wherein the results of these measurements are combined with the previous data. These checkups may be performed using the same or another device. Only the position and orientation of the patient need to be identical in order to enable the combined analysis of the data.

It is particularly advantageous when the recordings performed at a later point in time are exclusively optical or performed by means of ultrasound. Thus, no further X-ray recordings are performed in order to avoid further radiation exposure of the patient.

Through the integration of the X-ray imaging technique and the optical or ultrasound surface measurement, it becomes possible for a larger group of patients to benefit from the radiation-free surface measurement at checkups. The integrated method provides opportunities for advanced radiation-free analysis, both through static as well as functional imaging techniques (gait laboratory, running and movement analysis).

Based on the synchronous or quasi-synchronous measurement results, checkups are possible for patients with severe spinal deformities by using optical or ultrasound surface measurement, whereby the number of X-ray recordings are reduced, and thus the radiation dose and the risk of cancer may be reduced.

It is also favourable if the data obtained for the position and orientation of the vertebrae of the spinal column are used to determine the scoliosis angle. The so-called scoliosis angle is a three-dimensional generalisation of the Cobb angle, which often serves as a measure for assessing scoliosis. The general determination of the Cobb angle follows in that the neutral vertebrae are determined first of all. These are the vertebrae at the two turning points of the lateral curvature of the spine. A tangent is applied to the cover plates of the two neutral vertebrae in each case. The angle at which these tangents intersect is the Cobb angle. Instead of tangents, an alternative method uses two lines that are perpendicular to the cover plates of the upper and lower neutral vertebrae. The Cobb angle, however, always refers to a two-dimensional X-ray image, and thus does not take into account depth information. The scoliosis angle, however, takes into account all three spatial dimensions, so that, in addition to the lateral deflection of the spinal column, it also takes into account any existing sagittal bending and vertical twisting, and, therefore, represents a much more accurate measure for the assessment of scoliosis.

Preferably, the recording of surface data uses 3D video rasterstereography, or 4D video rasterstereography with an averaging technique, or the encoded light approach, or the phase shift method, or the line scanning method, or the time-of-flight method, or the ultrasound method, wherein (optical) 3D video rasterstereography is particularly preferred.

3D video rasterstereography is a three-dimensional light-optical imaging process, typically comprising a light projector and a video camera, and working on the principle of triangulation. In this case, the light projector projects parallel measurement lines or other projection patterns on an object in front of the measurement apparatus. The video camera records this, and transmits the data to a computer, which calculates the spatial representation of the object based on the deformation of the line pattern caused by the object. The camera and projector form two fixed points at a constant distance from one another. Likewise, the angles of the camera and projector lens with respect to one another are known. These constants enable all other distances and angles to be calculated simply, including the spatial position of each point on the projection surface. 3D video rasterstereography is employed mainly in medical environments as a radiation-free back measuring system.

4D video rasterstereography (see, for example, EP 1 718 206 B1) is a further development of 3D video rasterstereography that also uses the principle of triangulation. In fact, the fourth dimension is time, so that instead of individual images as in the case of 3D video rasterstereography, a sequence of images (a “film”) is recorded. The computer calculates the spatial representation of the object to be measured for each frame. As in the case of 3D video rasterstereography, 4D video rasterstereography is used in medical environments to measure the back. The image sequence recorded over a period of time enables further calculations, such as average calculations (4D with averaging) or function measurements, to be performed during movement of the patient.

In the case of the encoded light approach, a sequence of striped patterns is projected through a projector onto an object and recorded by a video camera. The sequence of the stripes is in accordance with the principle of binarisation, i.e. initially n parallel measuring lines, then n/2, then n/4 etc., are projected until only two lines are obtained. In this case, the recording of the images and the changing of the striped patterns in the sequence take place synchronously so that each image records a striped pattern of the sequence. The spatial representation of the measured object is calculated from the frames by triangulation.

Use of the method of the encoded light approach is limited by the resolving power of the stripe sensor. To increase the resolution further, the principle of the encoded light approach may be combined with the phase shift method. To this end, each stripe of the encoded light approach is represented at its highest resolution by using an intensity-modulated sawtooth signal. By modelling the scanned signal with a cosine function and determination of the phase position for the monitored point, the stripes of the encoded light approach may be resolved further.

In the case of the line scanning method, a single light stripe is projected onto an object and recorded by a video camera and transmitted to a computer for further calculation. The computer can calculate the spatial position of the stripe through triangulation. In order to detect an object wholly or partially, the stripe is passed over the object, whereby the computer then assembles the frames and calculates the spatial representation of the object. The line scanning method may be particularly well combined with the X-ray slot-recording technique.

In addition to conventional methods, the DLP (Digital Light Processing) projection technique may be used for the projection of different patterns. DLP was developed by Texas Instruments (TI) and registered as a projection technique brand, for example for video projectors and rear projection screens in home theatres and the presentation field, and under the name “DLP Cinema” in the digital cinema field.

In the case of the time-of-flight method (TOF), light pulses are directed at an object, and then recorded by a camera. Then the time needed for the light to reach the object and return (run-time measurement) is calculated for each pixel. The spatial representation of the measured object results from the total points.

A time of flight measurement also takes place in the case of the ultrasound method. In this case, ultrasound waves are directed at an object, and are then picked up again by a receiver. The time taken for the ultrasound waves to reach the object and return is measured and used to calculate the spatial representation.

Advantageously, the three-dimensional model is verified by the projection of the model on the at least one X-ray image. Should it be necessary, the model may also be improved iteratively in this manner.

The object is further solved by a device for determining the spatial position and orientation of the pelvis and/or the bone structures of the shoulder-arm region and/or the vertebrae of a spinal column of a vertebrate. This device comprises:

an X-ray recording device having an X-ray beam path;

an optical (i.e. with visible or infrared light) recording device to record surface data, wherein the optical recording device has an optical beam path;

as well as an optical element for superimposing the optical beam path and the X-ray beam path. For example, a deflecting mirror or prism may be used as an optical element.

Furthermore, the device comprises means for triggering both a recording with the X-ray recording device as well as with the optical recording device, such that the two recordings are separated by a maximum time interval, corresponding to the maximum X-ray recording time, of 1 second, preferably 0.5 seconds, particularly preferably 0.3 seconds, more preferably 0.1 seconds or most preferably 0.05 seconds;

Means for superimposing at least one of the recorded X-ray images and the optically obtained surface data; and

Means for calculating a three-dimensional model from the optically obtained surface data and the at least one X-ray image.

It is crucial in a recording method that both measurements are carried out simultaneously under similar or identical and reproducible geometric recording conditions, and thus a precise attitude and patient position form the basis of the two measurement results. To this end, it is necessary that the optical measurement system and the X-ray system are integrated. An absolutely synchronous examination procedure is optimal; a time-displaced examination procedure for both measuring methods is only acceptable if there is no appreciable change in the position of the patient between the two different recordings. Different magnification factors in the X-ray image may be calculated and corrected by the geometry known from the surface image. A technically flawless combination (matching) of the two imaging techniques is possible in this way.

All current conventional X-ray recording systems may form the basis for the method, insofar as they are suitable for taking pictures of the human skeleton. It is preferable that the X-ray recording device is an X-ray apparatus having large area image recording formats or image detectors, or an X-ray machine using a conventional film-screen recording technique, or an X-ray machine using a dose-reducing slot-recording technique.

On the image recording side, predominantly large-area detector systems with an area of up to 43 cm×43 cm are used for the radiography. This eliminates the conventional film-screen technique. In this way, all image results are immediately available in digital form, and image processing software allows optimisation of the image results. Formerly, a film was exposed in radiography. In order to reduce the X-ray dose for patients, film systems were developed, which also conventionally exposed a film, but where the X-ray dose per recording was significantly reduced. A large field of view of up to 43 cm×43 cm is exposed first in both recording techniques. The dose required is relatively high in both cases, since a scattered radiation is formed depending on the physical conditions of the body of a patient in relation to the irradiated volume. This scattered radiation can represent up to 90% of the total radiation which means, conversely, that only 10% of the radiation is imaged effectively when recording large volumes.

In the slot-recording technique, instead of a large-scale X-ray field, the X-rays are only applied through a slot in the form of a narrow striped image, whereby the slot traverses the body (scanning method). The total recording time amounts to several seconds. Only a small body volume is involved in each scan, thus the production of unwanted scattered radiation is largely avoided. This, combined with highly sensitive X-ray slot detectors, enables the X-ray dose to be reduced by a factor of 10 compared to conventional processes. The disadvantages of the slot-recording technique lie in the longer recording time required as well as in the limited applicability of the system to all parts of the body.

In addition, the optical recording device for recording surface data is preferably one which uses 3D video rasterstereography or 4D video rasterstereography with averaging technique, or the encoded light approach, or the phase shift method, or the line scanning method, or a time-of-flight method.

In a particularly preferred embodiment of the device, the optical recording device for recording surface data uses 3D video rasterstereography, and comprises the following additional components:

a) a light source that illuminates the optical beam path;

b) a mask or an arrangement of slot diaphragms, adapted to project an optical striped pattern on the spinal column region of the back of the vertebrate by means of the light source via the optical beam path; and

c) an optical detector, e.g. a digital camera, which is so displaced perpendicularly to the optical axis of the common part of the optical and X-ray beam paths, that it can record images of the striped pattern on the spinal column region of the back of the vertebrate.

Further particularly preferred is when the device further comprises means for performing the method described above. These include, inter alia, means for combining and documenting the measured results of the X-ray and optical recording devices, means for correcting the magnification factors of the X-ray images, means for superimposing the at least one X-ray image and the optically-obtained surface data and for generating a uniform true-to-scale representation, and means for calculating a three-dimensional model of elements of the bone structure from the optically-obtained surface data and the at least one X-ray image.

Further details and features will become apparent from the following description of preferred embodiments in conjunction with the dependent claims. In this way, the respective features may be implemented on their own or together in combination. The ways to solve the object are not limited to the embodiments. Thus including, for example, regional detail instead of all—not named—intermediate values and all conceivable subintervals.

The embodiments are shown schematically in the figures. The same reference numerals in the individual figures denote identical or functionally-identical elements or elements corresponding to one another in their functions. Specifically the figures show:

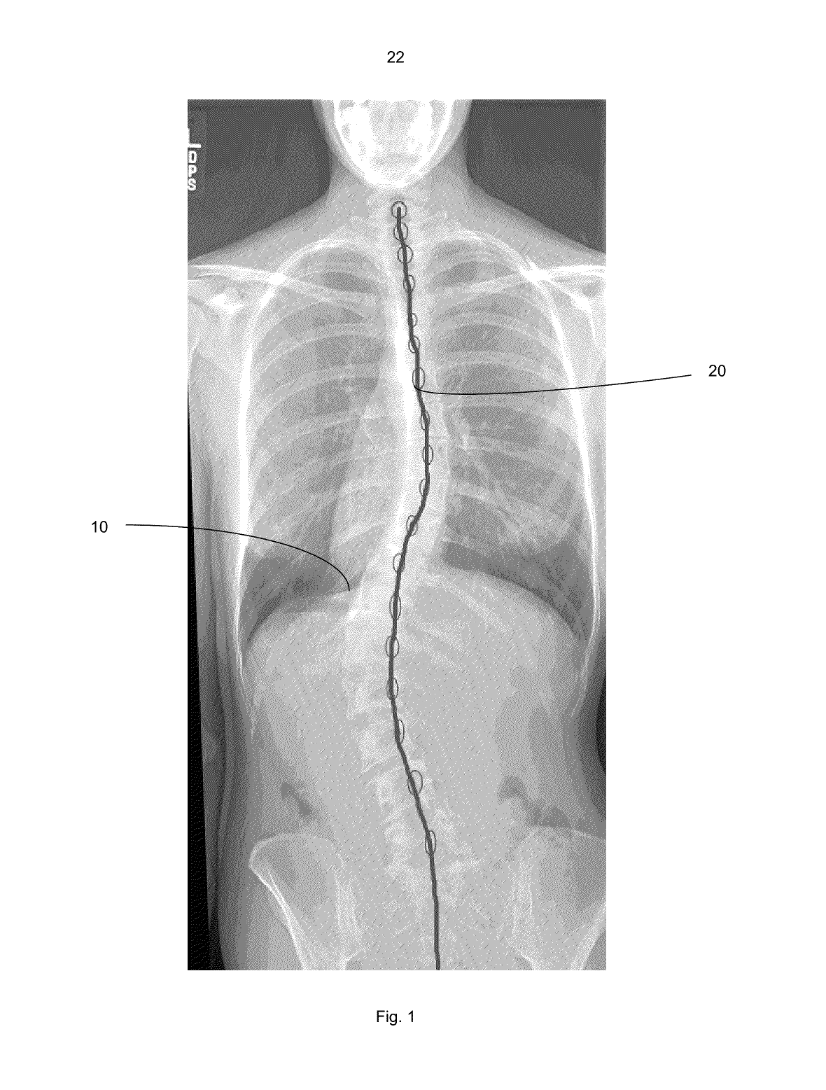

FIG. 1 shows selected elements of the bone structure of the spinal column in an X-ray image;

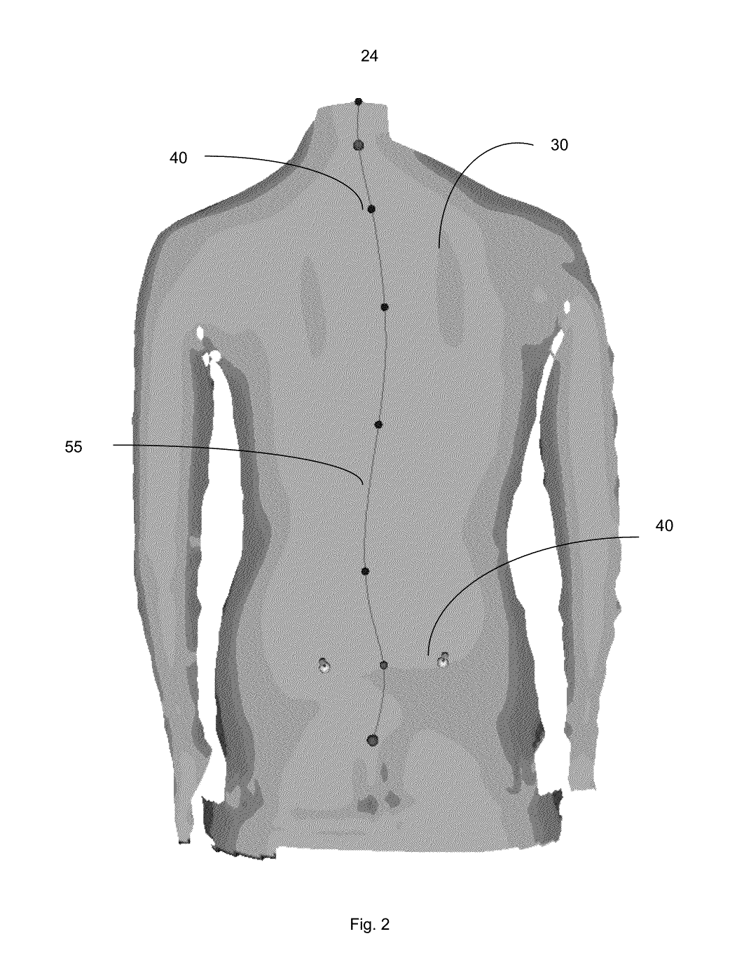

FIG. 2 shows distinct elements of the surface structure in the surface data of a human back;

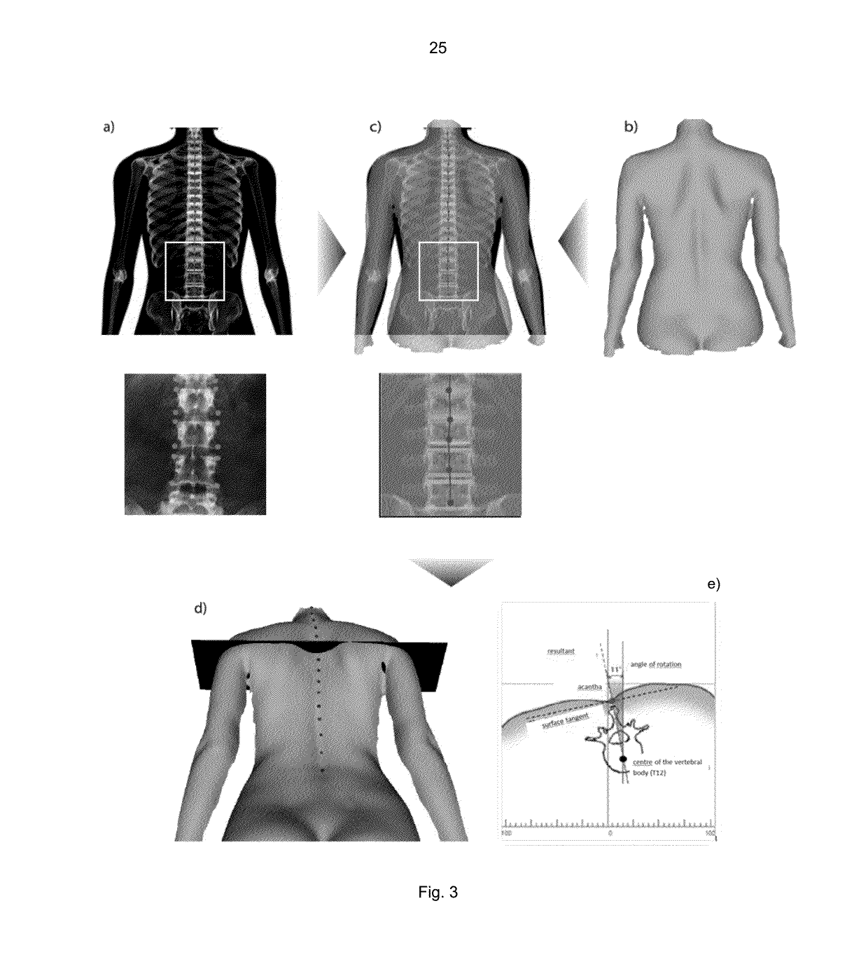

FIG. 3 shows an illustration of the determination of the orientation of the vertebrae of the spinal column;

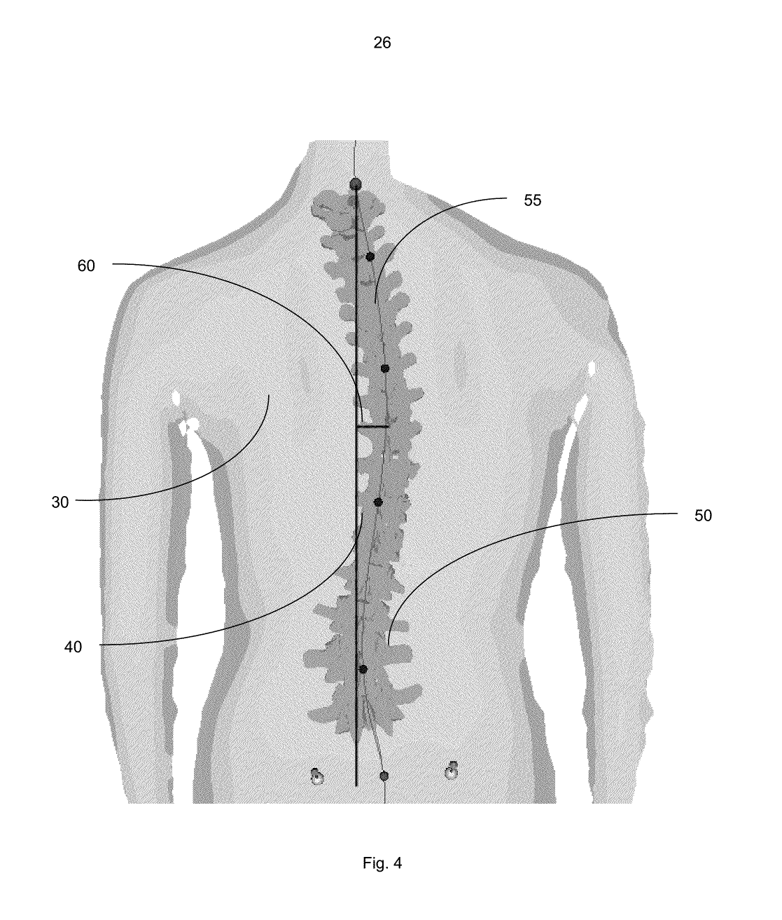

FIG. 4 shows a model of the spinal column, superimposed on the data surface of a human back;

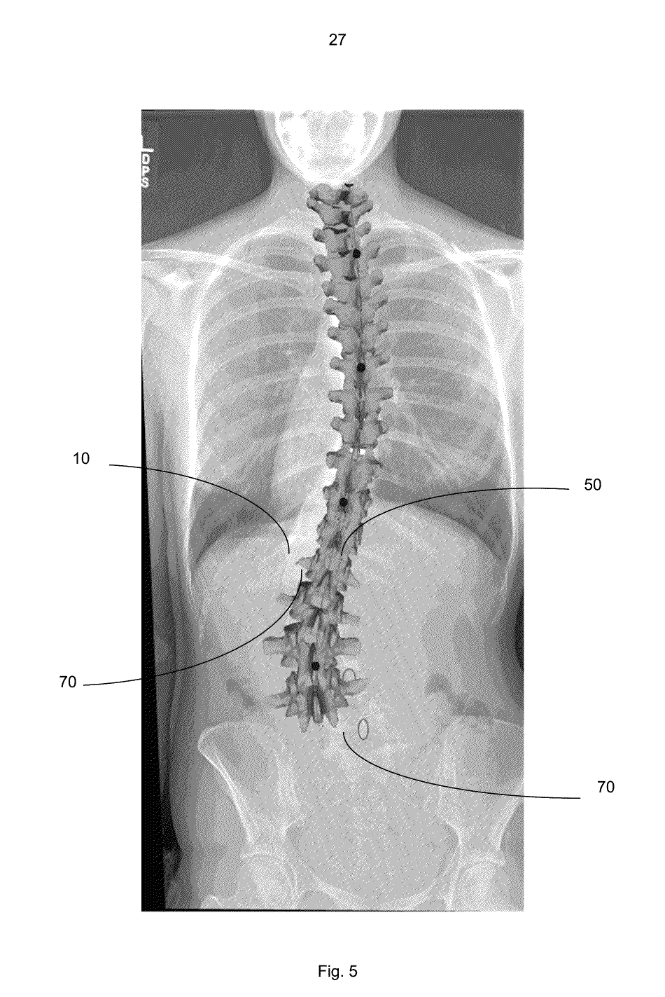

FIG. 5 shows a projection of a model of the spinal column on an X-ray image of the same.

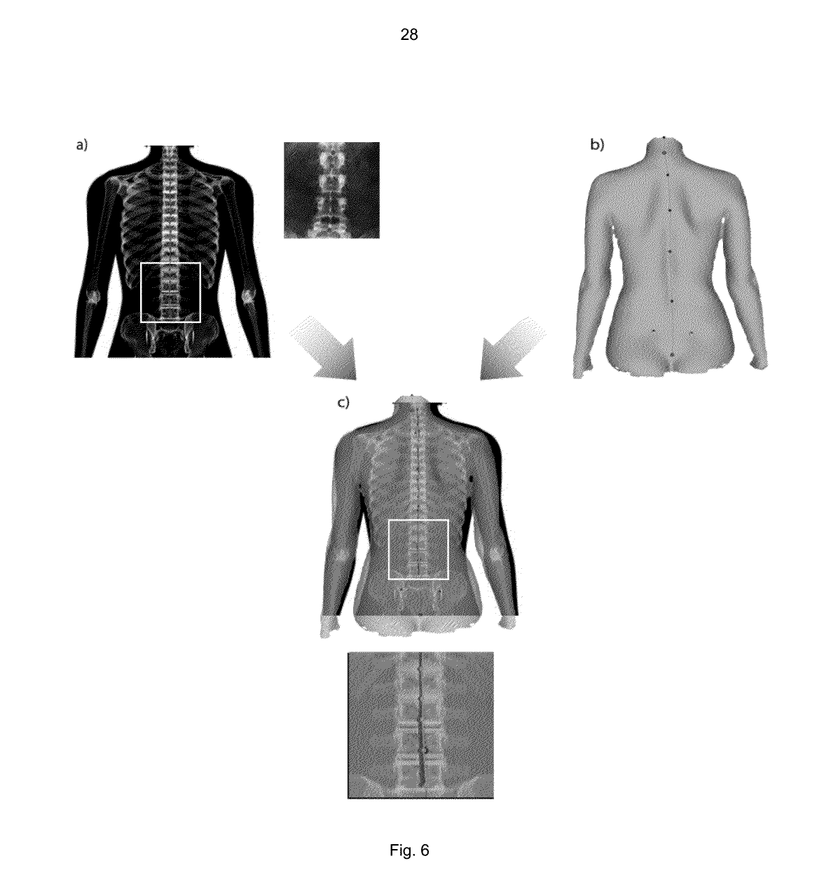

FIG. 6 shows a schematic representation of the sequence of a further part of the method according to the invention;

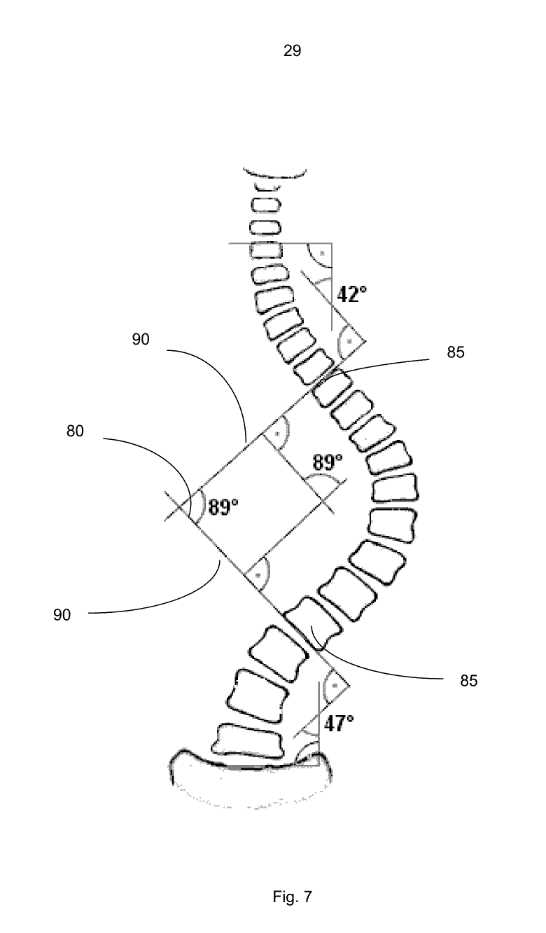

FIG. 7 shows a schematic representation of the Cobb angle (prior art); and

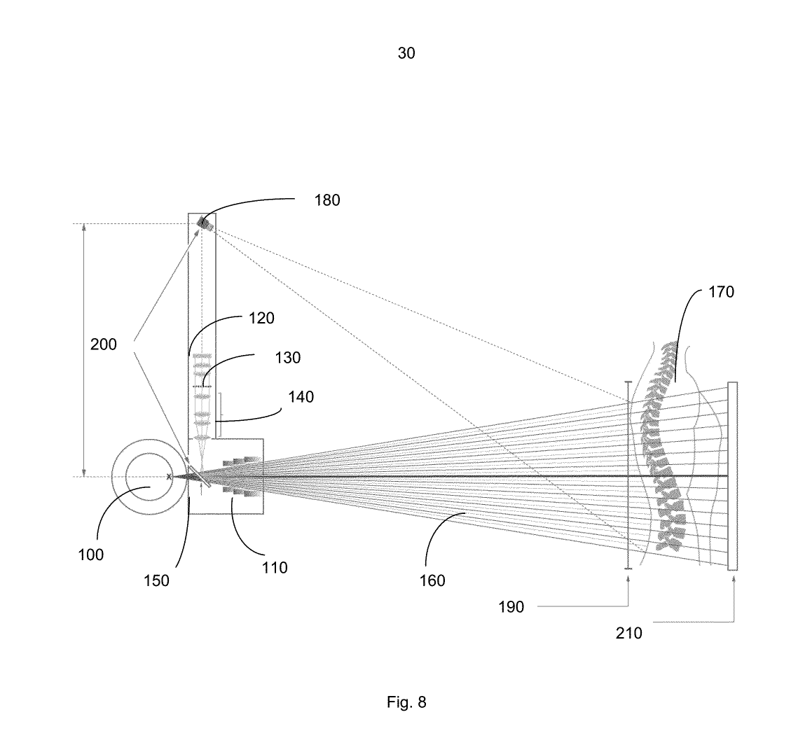

FIG. 8 shows a schematic representation of an embodiment of a device according to the invention.

According to the invention, to determine the spatial position and orientation, for example of the vertebrae of the spinal column of a person, at least one X-ray image is first recorded of at least a portion of the spinal column 10 (see FIG. 1). The location of elements of the bone structure is determined in this X-ray image. A selection of these elements of the bone structure, namely those can be determined in surface data of the back obtained by means of optical or ultrasound methods, are used as anatomical fixed points in the method according to the invention. Such a selection is, for example, the spinous processes 20 of the vertebrae of the spinal column. Where possible, the pedicles of the vertebrae of the spinal column are determined. The spinous process line is formed from the detected spinous processes 20.

Surface data 30 of at least a part of the back is recorded synchronously, i.e. with a typical time interval of 0.5 seconds maximum (see FIG. 2). This is performed by means of an optical (visible or infrared light) method or an ultrasound method. Preferably, three-dimensional video rasterstereography is used for this. Distinct elements are determined in the surface data; for example, the elevations that are caused by the tips of the spinous processes 20 of the vertebrae of the spinal column 10. To this end, the curvatures and symmetries of the surface data are calculated and balanced against known predetermined characteristics of the human back. The distinct elements in the surface data are typically to be found as extreme values or zero points of the curvature. A selection 40 of distinct elements is used as anatomical fixed points in order to infer the underlying bone structure, insofar as these elements of the bone structure can be determined on the X-ray image.

In FIG. 3, the recording and processing of the X-ray image is designated as a), while the recording of the surface data is designated as b). The X-ray recording and the surface data is superimposed on the basis of the anatomical fixed points (see FIG. 3 c)), wherein the X-ray image is scaled in advance as necessary, in order to obtain a uniform true-to-scale representation. Cutouts are delineated by white rectangles whose magnification is shown immediately below each cutout. After the superimposition, the determined spinous processes 20, as well as the resulting spinous process line from the X-ray image, are imaged on the 3D surface image, which is indicated in FIG. 3 as c).

A three-dimensional model 50 of the spinal column is calculated from the information obtained about the elements of the bone structure from the X-ray image and the surface data (see FIG. 4). Three orientation angles are determined for each vertebra, for example, from the position of the vertebrae and the position of the spinous processes. To this end, the section plane through the imaged spinous process is considered (see FIG. 3 d)). The surface profile in this section plane is mathematically determined, in order to calculate the orientation of the spinous process by calculation of the normal vector at this point, as shown in FIG. 3 e). The model therefore includes not only the position of the vertebrae and their exact orientation (sagittal, lateral as well as their rotation—this can only be insufficiently determined from the X-rays alone), and hence the overall curvature of the spinal column and the spinous process line 55, and in particular, for example, spinal column curvature caused by a scoliosis-related displacement 60.

The calculated three-dimensional model 50 of the spinal column 10 is projected on the X-ray image for verification, as shown in FIG. 5. In the case illustrated, deviations 70 between the projected model 50 and the X-ray image of the spinal column 10 can be seen. Therefore, improvements should be made to the parameters of the model. This is usually done iteratively until the projection of the model 50 of the spinal column 10 matches the X-ray image of the same, as closely as possible. Extant deviations may be used as a correction factor in checkups by means of a 3D surface measurement method (i.e. without an X-ray).

The determination of such a correction is shown in FIG. 6. In the X-ray recording, the spinous process line is formed as described above (shown in FIG. 6 as a)). The white box again identifies the enlarged cutout shown on the right of FIG. 6 a). The spinous process line is likewise determined in the synchronously recorded 3D surface data (see FIG. 6 b)). After superimposition of the X-ray image and the surface data, the two spinous process lines are compared; any differences occurring may serve as correction factors for future recordings using a surface measuring method (shown as c)). The white box again indicates the magnified cutout shown in FIG. 6 c).

Based on the calculated three-dimensional model of the spinal column, among other things, the scoliosis angle may be calculated. This is a three-dimensional generalisation of the known Cobb angle 80 whose determination is shown schematically in FIG. 7 (according to Skoliose-Info-Forum.de). Initially, the two neutral vertebrae 85, which, for example, form the turning points of the lateral curvature of the spinal column occurring in scoliosis, are determined. The angle, at which the tangents 90 applied to the cover plates of the neutral vertebrae intersect, is the Cobb angle 80. This is commonly used in the prior art as a measure for assessing scoliosis. In addition to the lateral curvature of the spinal column, the scoliosis angle also takes into account possibly existing sagittal bending as well as vertical rotation and is therefore a more accurate measure for the assessment of scoliosis.

A preferred embodiment of a device according to the invention is shown schematically in FIG. 8. This shows an X-ray tube 100, which emits the X-rays. The optical path is restricted by means of a lead aperture 110 so that it does not extend beyond the angle range to be imaged.

Further, a light source 120 is provided (typically, an LED is used) to illuminate a slot mask 130, so that an optical fringe pattern is created that is further imaged by projection optics 140. By means of the deflection mirror 150 (which is radiolucent), the optical beam path is combined with the X-ray beam path to form a common beam path 160. The striped pattern is projected on to the back of the patient 170. A digital video camera 180 is arranged perpendicularly displaced to this common beam path to record the optical recording field 190, so that triangulation 200 takes place. Behind the patient 170, there is a large-area X-ray detector 210. Because of the geometry of the beam path, means are also needed to scale the X-ray recording with respect to the optical data, or more precisely to reduce it (not shown).

Similarly, other regions of the body may be investigated on the same basis, i.e. the optical surface measurement, taking into account the radiographic determination of the bone structure. These include, in particular, the lower extremities (legs) and the shoulder-arm region.

### REFERENCE NUMERALS

10 Spinal column

20 Spinous processes of the vertebrae

30 Surface data of the human back

40 Distinct elements in the surface data

50 3D model of the spinal column

55 Spinous process line

60 Curvature through scoliosis

70 Deviation between model and spinal column

80 Cobb angle

85 Neutral vertebrae

90 Tangents to neutral vertebrae

100 X-ray tube

110 Lead aperture

120 Light source

130 Slot mask

140 Projection optics

150 Deflection mirror

160 Common beam path

170 Patient

180 Digital Video Camera

190 Optical recording field

200 Triangulation

210 X-ray detector

### CITED LITERATURE

### Cited Patent Literature

EP 1 718 206 B1 “Zeitabhängige dreidimensionale Muskel-Skelett-Modellierung auf Basis von dynamischen Oberflächenmessungen”

### Cited Non-Patent Literature

[1] Doody M. M., Lonstein J. E., Stovall M., Hacker D. G., Luckyanov N., Land C. E. (2000): “Breast Cancer Mortality After Diagnostic Radiography, Findings From the U.S. Scoliosis Cohort Study”, Spine, Volume 25: 2052-2063.
[2] Drerup B., Hierholzer E. (1987): “Automatic localization of anatomical landmarks on the back surface and construction of a body-fixed coordinate system”, Journal of applied Biomechanics 20, 961-970.
[3] Liljenqvist U., Halm H., Hierholzer E., Drerup B., Weiland M. (1998): “Die dreidimensionale Oberflächenvermessung von Wirbelsäulendeformitäten anhand der Videorasterstereographie”, Zeitschrift fur Orthopädie and ihre Grenzgebiete, Stuttgart [u.a.], Thieme, Vol. 136, No. 1, p. 57-64.
[4] Hackenberg L. (2003): “Stellenwert der Rückenformanalyse in der Therapie von Wirbelsäulendeformitäten”, Habilitationsschrift zur Erlangung der Venia Legendi für das Fach Orthopädie an der Westfälischen Wilhelms-Universität Münster.

1. A method for determining the spatial position and orientation of at least one of the pelvis, the bone structures of the shoulder-arm region. and the vertebrae of a spinal column of a vertebrate, comprising the following steps:
a) recording at least one X-ray image of at least a part of at least one of the spinal column, the pelvis, and the shoulder-arm region;
b) recording surface data of at least a part of a back of a vertebrate using an optical or ultrasound recording device;
c) wherein steps a) and b) take place with a maximum time interval of one second;
d) determining a position of elements of a bone structure using the X-ray image;
e) determining a position of distinct elements of a surface structure in the surface data;
f) wherein the position of elements of the bone structure is deduced from the position of the distinct elements of the surface structure;
g1) determining matching elements of the bone structure as anatomical fixed points from the at least one X-ray image and from the surface data; or
g2) determining anatomical fixed points using markers on the back of the vertebrate, wherein the markers are selected such that the markers are visible both in the surface data as well as on the X-ray image;
h) superimposing the at least one recorded X-ray image and the surface data using the anatomical fixed points; and
i) calculating a three-dimensional model of the elements of the bone structure from the surface data and the at least one X-ray image, wherein the model includes
i1) the position of the vertebrae and/or of the pelvis and/or
i2) the curvature of the spinal column and/or of the spinous processes and/or
i3) the orientation of the individual vertebrae and/or the pelvis and/or
i4) the location and orientation of the bone structures of the shoulder-arm region.

2. The method of claim 1, wherein the elements of the bone structure comprise:
a) the spinous processes and the pedicles of the vertebrae of the spinal column and/or
b) the sacrum, the upper edge of the ilium and the anterior and/or posterior superior Iliac spine and/or
c) the clavicle and the acromioclavicular joint and/or
d) the shoulder blades and/or
e) the shoulder blade edges.

3. The method of claim 1, wherein the distinct elements in the surface data are determined by analysing the surface properties, comprising the steps of
a) calculating curvatures and/or symmetries in the surface data; and
b) wherein the calculation of the curvatures and/or symmetries includes the fulfillment of certain predetermined conditions, which at least
b1) describe either the curvature or the symmetry of the surface, and
b2) describe either the relative position, bending, twisting or equidistance of the vertebrae of the spinal column.

4. The method of claim 1, further comprising:
a) scaling the at least one X-ray image; and
b) generating a uniform true-to-scale representation of the surface data and X-ray data.

5. The method of claim 1, further on comprising performing, at a later point in time, at least further optical or ultrasound recordings; and combining results of further optical or ultrasound recordings with the previous data.

6. The method of claim 5, wherein the recordings performed at a later point in time are carried out exclusively using an optical or ultrasound recording device.

7. The method of claim 1, further comprising determining a scoliosis angle using the data on the position and orientation of the vertebrae of the spinal column.

8. The method of claim 1, wherein the recording of surface data is performed using
a) 3D video rasterstereography, or
b) 4D video rasterstereography with averaging technique, or
c) the encoded light approach, or
d) the phase shift method, or
e) the line scanning method, or
f) the time-of-flight method, or
g) the ultrasound method.

9. The method of claim 1, further comprising verifying the three-dimensional model by projection of the model on the at least one X-ray image.

10. Device for determining the spatial position and orientation of the pelvis the bone structures of the shoulder-arm region, or the vertebrae of a spinal column of a vertebrate, comprising:
a) an X-ray recording device wth an X-ray beam path;
b) an optical recording device configured to record surface data, wherein the optical recording device has an optical beam path;
c) an optical element configured to superimpose the optical beam path and the X-ray beam path;
d) a triggering device that causes recordings with both the X-ray recording device and the optical recording device, such that the two recordings are made with a maximum time interval of one second;
e) a processor configured to superimpose the recorded at least one X-ray image and the optically obtained surface data, and
calculate a three-dimensional model from the optically obtained surface data and the at least one X-ray image.

11. The device of claim 10, wherein the X-ray recording device comprises
a) an X-ray machine with a large-area detector or
b) an X-ray machine using a conventional film-screen recording technique or
c) an X-ray machine using a slot-recording technique.

12. The device of claim 10, wherein the optical recording device for recording surface data uses
a) 3D video rasterstereography, or
b) 4D video rasterstereography with averaging technique, or
c) an encoded light approach, or
d) a phase shift method, or
e) a line scanning method, or
f) a time-of-flight method.

13. The device of claim 10. wherein the optical recording device for recording surface data uses 3D video rasterstereography and comprises the following additional components:
a) a light source that illuminates the optical beam path;
b) a mask, or an arrangement of slot diaphragms, adapted to project an optical striped pattern on the spinal column area of the back of the vertebrate using the light source via the optical beam path; and
c) an optical detector perpendicularly displaced relative to the optical axis of the common part of the optical and the X-ray beam paths, arranged so that the optical detector can record images of the striped pattern on the spinal column region of the back of the vertebrate,

14. (canceled)
